(19) United States
(12) Patent Application Publication
(10) Pub. No.: US 2017/0343570 A1
(43) Pub. Date: Nov. 30, 2017

(54) MT-45 IMMUNODETECTION

(71) Applicant: RANDOX LABORATORIES LTD., Crumlin (GB)

(72) Inventors: ELOUARD BENCHIKH, CRUMLIN (GB); IVAN MCCONNELL, CRUMLIN (GB); PHILIP LOWRY, CRUMLIN (GB); PETER FITZGERALD, CRUMLIN (GB)

(21) Appl. No.: 15/610,199

(22) Filed: May 31, 2017

(30) Foreign Application Priority Data
May 31, 2016 (GB) 1609473.2

(51) Int. Cl. G01N 33/94; C07K 16/06; C07D 333/32; C07D 295/096; C07K 14/765; C07K 14/47; C12N 9/08; C07K 16/44

(52) U.S. Cl. CPC G01N 33/9486; C12Y 111/01007; C12N 9/0065; C07K 16/06; C07K 16/44; C07D 295/096; C07K 14/765; C07K 14/47; C07D 333/32; C07K 2317/33

(57) ABSTRACT

Components for enabling immunodection of MT-45 are described including immunogens, antibodies derived from the immunogens, immunoassay methods, detecting agents and kits.

### CROSS-REFERENCE TO RELATED APPLICATIONS

The instant application claims the benefit of priority under 35 USC $ 119 to United Kingdom Application No. 1609473.2, entitled “MT-45 IMMUNODETECTION” filed May 31, 2016, the entire contents of which are incorporated herein by reference in their entirety.

### BACKGROUND OF THE INVENTION

MT-45, systematic name (R,S)-1-cyclohexyl-4-(1,2-diphenylethyl)piperazine, is a synthetic drug of abuse of the opioid class, the S-enantiomer having high affinity for delta and kappa opioid receptors. It has therapeutic origins and was a candidate analgesic drug having a potency similar to morphine but has since been detected in several biological samples of individuals subject to fatal and non-fatal intoxication (reference 1). Given the emergence of this toxic drug of abuse and its on-going legislative control, methods for its detection in biological and pre-ingested samples are required. Gas chromatography (GC) and Liquid chromatography (LC) linked to mass spectrometry (MS), Fourier Transform infrared spectroscopy and proton and carbon-13 nuclear magnetic resonance spectroscopy have been described for the detection of MT-45 (reference 2). Papsun (2016) describes detection and quantification of MT-45 in blood samples using LC-MS-MS. These techniques require specialist staff for their operation, are expensive and are not amenable to use outside of the laboratory.

### SUMMARY OF THE INVENTION

Described are the first known immunogens and antibodies for MT-45. The invention further describes detecting agents and methods and kits comprising the antibody.

In one embodiment, the present invention is an immuogen of structure

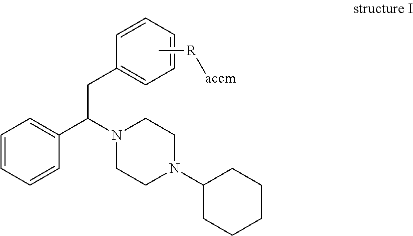

wherein R is a spacing group attached to the ortho, meta or para position of the phenyl ring and to an antigenicity conferring carrier material (accm).

In another embodiment, the present invention is an antibody raisable against an immunogen of structure III:

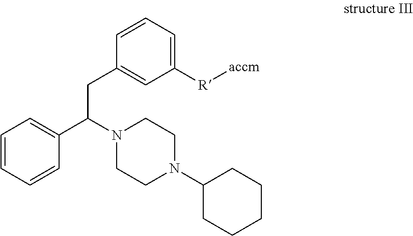

wherein: R′ is -(Q′)ₘ-Y′—X′; m is 1; Q′ is —O—; Y′ is —CH₂1'; X′ is carboxy; and the accm is keyhole limpet haemocyanin, wherein the antibody has an IC₅₀ of <1.00 ng/ml to MT-45 as calculated from a standard curve using MT-45-CME-HCTL-maleimide-HRP (Tracer 2) as detecting agent.

In yet another embodiment, the present invention is an immunoassay method of detecting or determining MT-45 in an in vitro sample or in a solution comprising: i) contacting the sample or solution with a detecting agent and an antibody of any one of claims 7-9; detecting the amount of detecting agent bound to the antibody; and deducing the presence or amount of MT-45.

In yet another embodiment, the present invention is a detecting agent of structure

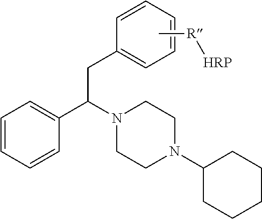

wherein R″ is a crosslinking group attached to the meta or para position of the phenyl ring and HRP is horseradish peroxidase.

### BRIEF DESCRIPTION OF THE DRAWINGS

FIG. 1 shows the structures of MT-45 Hapten A and Hapten B;

FIG. 2 shows the synthesis of Hapten A;

FIG. 3 shows the synthesis of (R,S)-1-cyclohexyl-4-[2-(4-carboxymethoxyphenyl)-1-phenylethyl]piperazine hapten;

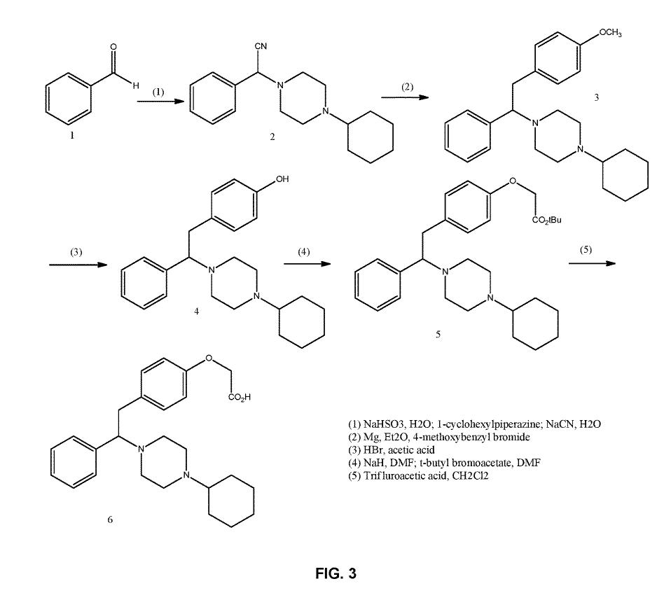

FIG. 4 shows the structures of the Immunogens from Hapten A;

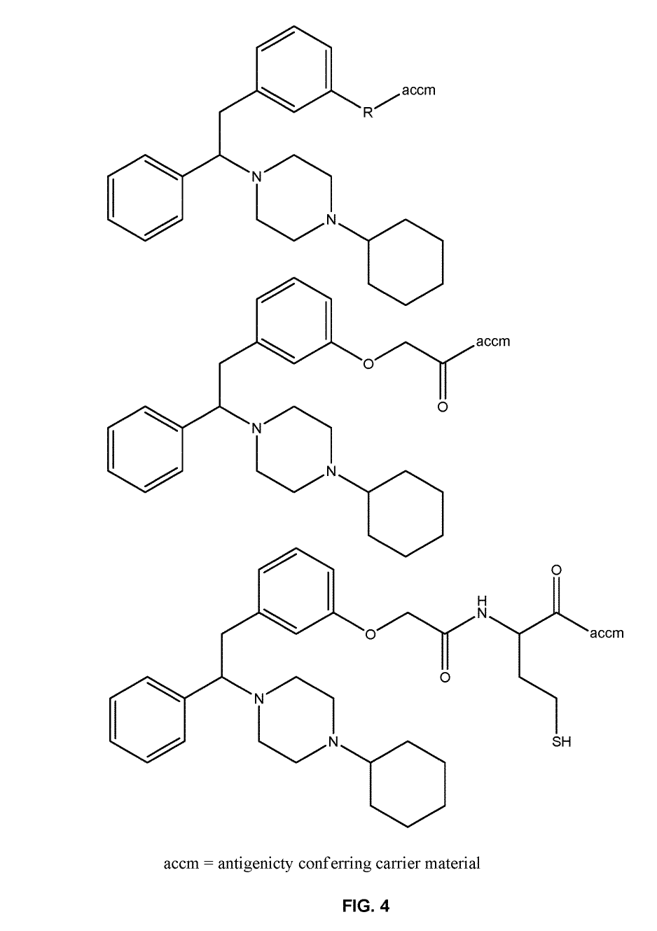

FIG. 5 shows the structures of the Immunogens based on derivatisation of the 4-position of the phenyl moiety attached to the 2-ethyl position of MT-45; and

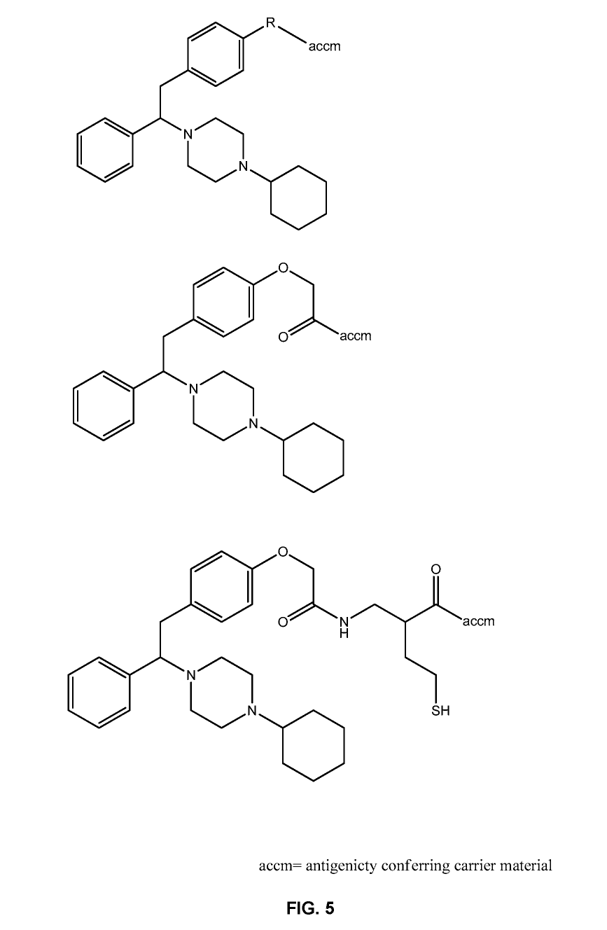

FIG. 6 shows the structures of the detecting agents (tracers).

### DETAILED DESCRIPTION OF THE INVENTION

In a first aspect, the invention describes an immunogen of structure I.

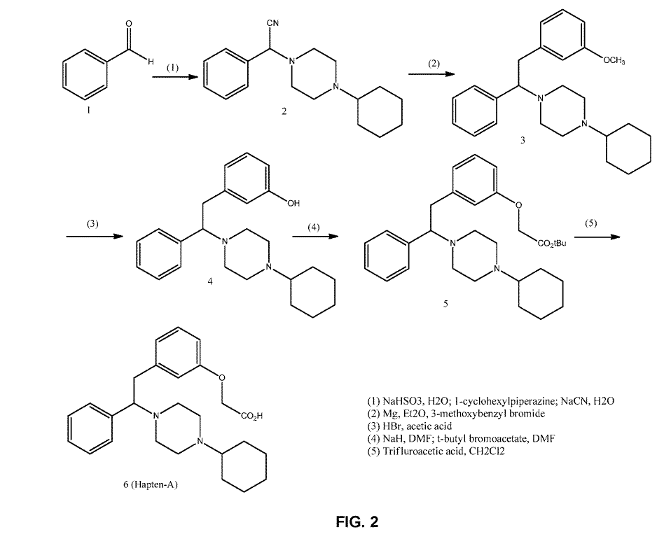

### Phenyl Ring 1

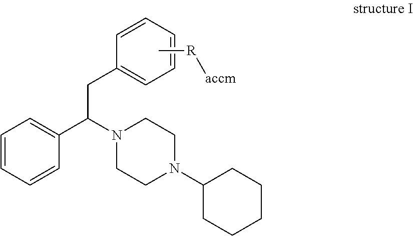

in which R is a spacing group attached to the meta, ortho or para position of the phenyl ring (phenyl ring 1) and the accm is an antigenicity conferring carrier material; R is preferably attached to the meta or para position. The spacing group (also known as a crosslinker or crosslinking group) is standard in the art and can be any appropriate acyclic or cyclic system or a combination of cyclic and acyclic systems that provides a means to both link and impart a degree of separation between the small molecule (in this case MT-45) to which antibodies are to be raised and the accm. For example, it can be a short chain saturated or unsaturated, substituted or unsubstituted alkanediyl or alkenediyl chain of 1-10 carbon atoms, or arylene groups, or saturated or unsaturated cycloalkane(s), or heterocycle(s) or combinations of alkanediyl, alkenediyl, arylene groups, usually supporting a functional group (e.g. carboxy, amino, carbonyl, ester) at one or both of the chain or ring end(s), following or preceding attachment to the phenyl ring and the accm i.e. the spacing group is made up of atoms that can be already part of MT-45 or a MT-45 analogue, or can be added to MT-45 or a MT-45 analogue through chemical synthesis, or can be a combination of both e.g. see FIGS. 2 & 3 and Examples 5 and 6 in which an MT-45 analogue is produced incorporating an oxygen atom (part of spacing group) on phenyl ring 1 during its synthesis prior to addition of a carboxymethylene moiety (part of spacing group). The total linear chain length of the crosslinker is preferably 1-10 atoms, most preferably 1-6 atoms. In the context of the invention, the phrase ‘total linear chain length’ in conjunction with 1-10 atoms or 1-6 atoms, implies that if a ring system is present in the crosslinker it is afforded the value of one atom i.e. a benzene ring and a cyclohexane ring in the crosslinker corresponds to 2 atoms and diphenylmethane (phenyl-CH₂-phenyl) corresponds to 3 atoms. For the invention, R is preferably -(Q)m-Y—X—, in which Q or Y (if Q is not present) is attached to the meta or para position of the phenyl ring and m=0 or 1; Y can comprise a substituted or unsubstituted, straight or branched chain alkanediyl moiety, a substituted or unsubstituted, saturated or unsaturated cycloalkylene moiety or a substituted or unsubstituted arylene moiety; X, before attachment to the antigenicity conferring carrier material, is chosen from carboxy (—CO₂H), dithiopyridyl, maleimidyl, amino (—NH₂), hydroxyl (—OH), thiol (—SH), thioester (—C(O)—S—R⁻ or —C(S)—O—R⁻, in which R⁻=alkyl) or aldehyde (—C(O)—R⁺, in which R⁺ is —H or alkyl); Q is —O—, —S—, —NH—, —C(O)—, —C(O)—O—., —S(O)—, —S(O)₂—, —C(S)—O— or —C(O)—S—. In a preferred embodiment of the invention, —(Q)m-Y—X— comprises: Y═C₁₋₆, substituted or unsubstituted, straight chain, saturated alkanediyl moiety; m=1 and Q is —O—, —NH— or —S—; X=—C(O)—, —C(O)—O—, —O—C(O)—, —C═N—, —NH—, —S—, —S(O)—, —S(O)₂—, —S—C(O)—, or —C(O)—S—; preferably m=1; Q is —O—, —NH— or —S—; and Y is a C₁₋₆ substituted or unsubstituted, straight chain, saturated alkanediyl moiety; more preferably, m=1; Q is —O—; Y is —CH₂—; and X is carboxy. In one embodiment R is —O—CH₂—CO—. In one embodiment R is —O—CH₂—CO— (when using EDC for synthesis of immunogen and detecting agent).

The term “alkanediyl” and “alkylene” are interchangeable and as used herein, means a saturated divalent straight or branched chain hydrocarbon group and is exemplified by methylene, ethylene, isopropylene and the like.

The term “cycloalkylene” as used herein, means a saturated or unsaturated divalent carbon only containing ring system, having three to fourteen ring carbon atoms. In some embodiments, the number of carbon atoms is 3 to 10. In other embodiments, the number of carbon atoms is 4 to 7. In yet other embodiments, the number of carbon atoms is 5 or 6. The term includes monocyclic, bicyclic or polycyclic, fused, spiro or bridged cycloalkylene ring systems. The term also includes polycyclic ring systems in which the cycloalkylene ring can be “fused” to one or more non-aromatic carbocyclic or heterocyclic rings or one or more aromatic rings or combination thereof, wherein the radicals or points of attachment are on the cycloalkylene ring. “Fused” bicyclic ring systems comprise two rings which share two adjoining ring atoms. Bridged bicyclic group comprise two rings which share three or four adjacent ring atoms. Spiro bicyclic ring systems share one ring atom. Examples of cycloalkylene groups include, but are not limited to, cyclohexyl, cyclopropyl, and cyclobutyl.

The term “arylene” as used herein, means an aromatic divalent carbon only or heteroaromatic ring system, having five to fourteen ring atoms. In some embodiments, the number of atoms is 5 or 6. The term includes monocyclic, bicyclic or polycyclic, fused, spiro or bridged arylene ring systems. The term also includes polycyclic ring systems in which the arylene ring can be “fused” to one or more non-aromatic carbocyclic or heterocyclic rings or one or more aromatic rings or combination thereof, wherein the radicals or points of attachment are on the arylene ring. “Fused” bicyclic ring systems comprise two rings which share two adjoining ring atoms. Bridged bicyclic group comprise two rings which share three or four adjacent ring atoms. Spiro bicyclic ring systems share one ring atom.

As described herein, the immunogens may optionally be substituted with one or more substituents, such as illustrated generally below, or as exemplified by particular species of the invention. It will be appreciated that the phrase “optionally substituted” is used interchangeably with the phrase “substituted or unsubstituted.” In general, the term “substituted”, whether preceded by the term “optionally” or not, refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position. When the term “optionally substituted” precedes a list, said term refers to all of the subsequent substitutable groups in that list.

Selection of substituents and combinations of substituents envisioned by this invention are those that result in the formation of stable or chemically feasible compounds. The term “stable”, as used herein, refers to compounds that are not substantially altered when subjected to conditions to allow for their production, detection, and, specifically, their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week. Only those choices and combinations of substituents that result in a stable structure are contemplated. Such choices and combinations will be apparent to those of ordinary skill in the art and may be determined without undue experimentation.

Suitable substituents on a saturated or unsaturated carbon of an alkyl, cyclalkyl, or aryl ring are C₁-C₆ alkyl, halogen, cyano, oxo, —NCO, —ORb, —SR^{b}, —S(O)R^{a}, —SO₂Ra, —NR^{b}R^{c}, —C(O)R^{b}, —C(O)OR^{b}, —OC(O)R^{b}, —NRC(O)R^{b}, —C(O)NR^{b}R^{c}, —NR^{b}C(O)NR^{b}R^{c}, —NR^{b}C(O)OR^{b}, —OCONR^{b}R^{c}, —C(O)NRCO₂R^{b}, —NR^{b}C(O)NR^{b}C(O)OR^{b}, —C(O)NR(OR^{b}), —SO₂NR^{c}R^{b}, —NR^{b}SO₂R^{b}, —NR^{b}SO₂NR^{c}R^{b}, or —P(O)(OR^{a})₂—; or two substituents join together with the atoms to which they are attached to form a 5-7-membered cycloalkyl or heterocyclic ring. Each R^{a}, R^{b} and R^{c} are each independently —H or C₁-C₆ alkyl. Other suitable substituents for a saturated carbon of an alkyl, or cycloalkyl, include the following: ═O, +S, ═NNHR*, ═NN(R*)₂, ═NNHC(O)R*, ═NNHCO₂(alkyl), ═NNHSO₂(alkyl), or ═NR*, wherein each R* is independently selected from —H or C₁-C₆ alkyl.

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this invention, unless only one of the isomers is drawn specifically. As would be understood to one skilled in the art, a substituent can freely rotate around any rotatable bonds. Therefore, single stereochemical isomers as well as enantiomeric, diastereomeric, cis/trans, conformational, and rotational mixtures of the present compounds are within the scope of the invention.

Unless otherwise indicated, all tautomeric forms of the compounds of the invention are within the scope of the invention.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays. Such compounds, especially deuterium analogues, can also be therapeutically useful.

The compounds of the invention are defined herein by their chemical structures and/or chemical names. Where a compound is referred to by both a chemical structure and a chemical name, and the chemical structure and chemical name conflict, the chemical structure is determinative of the compound's identity.

In a further preferred embodiment R (Q or Y), as described in the previous immunogen embodiments of the invention, is attached to the meta position of the phenyl ring (phenyl ring 1). Antigenicity conferring carrier materials are well known and well-established in the art and can be any material that makes all or part of the hapten immunogenic, such as a protein, a protein fragment, a synthetic polypeptide or a semi-synthetic polypeptide. The most commonly used and most preferable accmₛ of the current invention are keyhole limpet haemocyanin (KLH), bovine thyroglobulin (BTG), bovine serum albumin (BSA), egg ovalbumin, bovine gamma globulin or cationised BSA (see General Methods, Examples and Results for further examples). The process of immunogen formation generally involves coupling of a hapten to a crosslinking agent, the latter subsequently coupled to an accm. Alternatively, the crosslinking agent can be coupled to the accm in the first instance and subsequently couples to the hapten. In both these processes the crosslinking agent generally incorporates at least two reactive functional groups. Numerous crosslinkers and accms are commercially available and have been described in the literature (Thermo Scientific Crosslinking Technical Handbook, 1606073 04/2009; Bioconjugate Techniques G. Hermanson, ed, Academic Press, 1996, 785pp).

### Immunoassay Method

The invention further describes an immunoassay method of detecting or determining MT-45 in an in vitro sample or in a solution comprising contacting the sample or solution with a detecting agent and an antibody derived from an immunogen of the invention, detecting the bound detecting agent and deducing the presence or amount of MT-45. In one embodiment, the detecting agent competes with MT-45 present in the sample for binding to the antibody. By ‘detecting’ is meant qualitatively analysing for the presence or absence of a substance; by ‘determining’ is meant quantitatively analysing for the amount of a substance present. The detecting agent can be any molecule which can bind to an antibody capable of binding to MT-45. The detecting agent when added to a sample competes with MT-45 present in the sample for binding to the antibody. The detecting agent is preferably of structure II

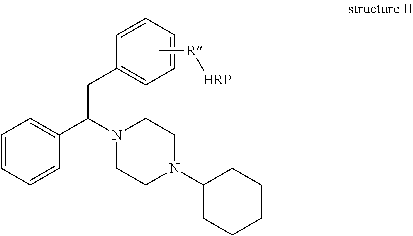

in which R″ is a crosslinking group attached to either the meta or para position of the phenyl ring and HRP is horseradish peroxidase. R″ is -(Q)m-Y—X— as described above for R including all embodiment described herein. Examples of possible detecting agents of the invention are given in Examples 9 and 10 of the General Methods, Examples and Results section. The antibodies described herein were derived from immunogens produced from haptenic racemic mixtures; however, in one embodiment using asymmetric synthesis or enantiomeric separation, the (S) or (R) enantiomer haptens can also be used for immunogen formation. The term “hapten” refers to small molecule pre-immunogen precursors e.g. molecule 6 of FIG. 2. The detection or determination step is usually qualified or quantified with the aid of a calibrator. A calibrator is well known in the art and enables a threshold concentration or the exact or calibrator equivalent amount of an analyte to be determined. A calibrator curve or threshold concentration may be suitable for several analytes or may have to be derived for each individual analyte. The determination of an exact or calibrator equivalent amount of an analyte often requires the construction of a calibration curve (also known as a standard curve). The number of calibrator points can vary, but is usually from 5 to 9.The in vitro sample is any suitable biological sample such as, but not limited to, blood, serum, plasma, urine or saliva.

The in vitro sample is preferably a serum, plasma or urine sample. The solution can be a liquid suspected of containing MT-45. Alternatively, as MT-45 is often in solid form, analysis may require pre-treatment to achieve a formulation suitable for immuno-analysis, such as dissolution in a suitable liquid.

The immunoassay method is preferably based on the well-known competitive assay format in which a target analyte which binds to the antibody i.e. the molecule to be detected or determined, competes with a detecting agent which also binds to the antibody, for binding sites on the antibody. The detecting agent can be any substance which leads to a detectable or measurable signal and typically incorporates an enzyme which promotes light emission from a substrate, a fluorophore or a radioactive label; it is usual for an immunoassay that the detecting agent incorporates a structure similar to the target analyte to which an enzyme or a substance having fluorescent properties has been conjugated, or in which a radiolabel has been incorporated. Preferably, for the immunoassay method of the invention, the detecting agent is based on a compound with structure II. Conjugation of R is by way of standard methods familiar to the skilled person and may involve the crosslinking methodology and groups described previously for the immunogen of the invention.

For purposes of comparison, one analyte with high cross-reactivity is generally given a value of 100%, with all other analytes accorded a value relative to this; in addition, as is known by one skilled in the art, for cross-reactivity to be of practical use the analyte specific antibody must display a high sensitivity as measured by a suitable metric such as the IC₅₀. The IC₅₀ is a commonly used indicator of antibody sensitivity for immunoassays.

The invention further describes an immunoassay method for detecting MT-45 in a solution or an in vitro sample from a subject, the method comprising contacting a detecting agent and an antibody of the invention with the solution or in vitro sample and detecting the presence and/or amount of MT-45, characterised in that the antibody specific to MT-45 has a cross-reactivity of between about about 60% to about 95%, preferably between about 70% to about 95%, more preferably between about 75% to about 90% for 3-hydroxy MT-45 compared with about 100% cross-reactivity to MT-45; most preferably the antibody has a cross-reactivity of less than about 90% for 3-hydroxy MT-45 compared with about 100% cross-reactivity to MT-45. In one embodiment, the antibody has a cross-reactivity of about 83%, about 81% or about 89% for 3-hydroxy MT-45 compared with about 100% cross-reactivity to MT-45. In one embodiment, the antibody has a cross-reactivity of 83%±10%, 81%±10% or 89%±10% for 3-hydroxy MT-45 compared with 100%±10% cross-reactivity to MT-45. In one embodiment, the antibody has a cross-reactivity of 83%±5%, 81%±5% or 89%±5% for 3-hydroxy MT-45 compared with 100%±5% cross-reactivity to MT-45.

As used herein, “about” is to account for such instances as slight measurement variations which occur within scientific measurement due to inter-individual and equipment variation in each scientific procedural step, as well as mathematically based variation in result reporting which can incorporate numerical ‘rounding-down’ or ‘rounding-up’ operations. For example, about means a variation of ±10%, ±8%, ±5%, ±2%, preferably ±10%.As used herein, an antibody “specific” to a particular molecule shows the greatest binding to that molecule compared to other molecules as measured by a recognised metric such as the limit of detection, limit of quantification or the IC₅₀.

The term “subject” refers to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In one embodiment, the subject is a non-human animal such as a farm animal (e.g., a horse, cow, pig or sheep), or a pet (e.g., a dog, cat, guinea pig or rabbit). In another embodiment, the subject is a “human”.

### Antibodies

The invention further describes an antibody derived or are raisable from an immunogen of structure I. Preferably, the immunogen from which the antibody is derived has the spacing group attached to the meta or para position of the phenyl ring (phenyl ring 1) of MT-45. More preferably the antibody is derived from an immunogen of structure III

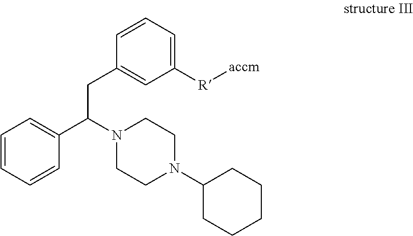

in which R′ is -(Q)_{m′}-Y′—X′—, in which Y′ is C₁₋₆ substituted or unsubstituted, straight chain, saturated alkanediyl moiety; m′=1 and Q′ is —O—, —NH— or —S—; and X′, attached to the accm, is chosen from —C(O)—, —C(O)—O—, —O—C(O)—, —C═N—, —NH—, —S—, —S(O)—, —S(O)₂—, —S—C(O)—, or —C(O)—S—. Most preferably, the antibody is derived from an immunogen in which Q′ is —O—, Y′ is —CH₂—, X′ is carboxy and the accm is keyhole limpet haemocyanin. There are several parameters that can be used to compare the relative degree of binding of an antibody to different analytes including the lowest limit of detection, the lowest limit of quantification and the IC₅₀. The IC₅₀ is determined from a standard curve which is generated using a competitive assay (see Example 12 of the General Method, Examples and Results section and Table 1) and can be used to derive analyte cross-reactivities. To enable an assay to be effectively applied, an IC₅₀ of less than or about 50 ng/ml, preferably less than or about 10 ng/ml, more preferably less than or about 5 ng/ml or less than or about 1 ng/ml, for any individual analyte is preferred. Derivation of these parameters enables the relative cross-reactivities of various analytes to an antibody to be determined. In another embodiment the antibody of the invention has an IC₅₀ of about less than or about 20 ng/ml, more preferably less than or about 10 ng/ml, most preferably less than or about 1 ng/ml to MT-45. Preferably, the antibody is derived from an immunogen of structure III (structure III preferably has Q′=—O—, —NH— or —S—, Y′=C₁₋₆ substituted or unsubstituted, straight chain, saturated alkanediyl, X′=—C(O)—, —C(O)—O—, —O—C(O)—, —C═N—, —NH—, —S—, —S(O)—, —S(O)₂—, —S—C(O)—, or —C(O)—S—) and has an IC₅₀ of <1.00 ng/ml to MT-45 as calculated from a standard curve using MT-45-CME-HCTL-maleimide-HRP (Tracer 2) as detecting agent. More preferably the antibody is derived from an immunogen in which Q′ is —O—, Y′ is —CH₂—, X′ is carboxy and the accm is keyhole limpet haemocyanin and has an IC₅₀ of <1.00 ng/ml to MT-45 as calculated from a standard curve using MT-45-CME-HCTL-maleimide-HRP (Tracer 2) as detecting agent. Due to inter-molecular attractive forces such as hydrogen bonding and van der Waal's forces there is often a degree of binding or affinity between two molecules whatever their respective structures; the skilled person recognizes that no cross-reactivity or minimal cross-reactivity implies that in the context of a working immunoassay any binding or interaction between an antibody and non-target analytes is at such a low level that it does not compromise the integrity of the immunoassay i.e. false positives, whether qualitative or quantitative, are avoided.

In one embodiment, the antibodies of the present invention have a cross-reactivity of between about about 60% to about 95%, preferably between about 70% to about 95%, more preferably between about 75% to about 90% for 3-hydroxy MT-45 compared with about 100% cross-reactivity to MT-45; most preferably the antibodies have a cross-reactivity of less than about 90% for 3-hydroxy MT-45 compared with about 100% cross-reactivity to MT-45. In one embodiment, the antibody has a cross-reactivity of about 83%, about 81% or about 89% for 3-hydroxy MT-45 compared with about 100% cross-reactivity to MT-45. In one embodiment, the antibody has a cross-reactivity of 83%±10%, 81%±10% or 89%±10% for 3-hydroxy MT-45 compared with 100%±10% cross-reactivity to MT-45. In one embodiment, the antibody has a cross-reactivity of 83%±5%, 81%±5% or 89%±5% for 3-hydroxy MT-45 compared with 100%±5% cross-reactivity to MT-45.

The invention also describes an antibody which binds specifically to M-45 and shows between about about 60% to about 95% cross-reactivity to 3-hydroxy MT-45 compared to a cross-reactivity of about 100% to MT-45. The antibody is further characterised by having an IC₅₀ of less than or about 1.0, 0.8, 0.5 or 0.3 ng/ml to MT-45.

The antibodies of the invention can be used in the immunoassay methods and kits of the invention as described herein.

The term “antibody” as used herein refers to an immunoglobulin or immunoglobulin-like molecule. In a one embodiment, the antibodies are monoclonal or polyclonal antibodies. However, the skilled person will understand that any type of immunoglobulin molecule or fragment thereof can be used, for example, Fab fragments, scFv fragments and any other antigen binding fragments all of which fall within the scope of the current invention. The antibodies may be produced by any method as known to those skilled in the art. Any suitable host animal may be used in the immunisation process including a mammalian animal for example, but not limited to, sheep, rabbit, mouse, guinea pig or horse. In addition, the antibodies may be in the form of polyclonal antisera.

The term “raisable” means that the antibody can be raised from an immunogen of of the current invention but is not necessarily so raised. In this context, “raisable” includes, but is not limited to, “raised”. In relation to the antibodies described herein, in the context of the current invention, “raised” is synonymous with “derived”.

The phrase “an antibody which binds or specifically binds to an epitope of structure . . . ” implies that the antibody, if polyclonal, will comprise clones whose high concentration and binding characteristics ensure an assay incorporating the antibody will bind to and ultimately support the identification of the compound of interest. Alternatively, the antibody could be a monoclonal antibody specific for a particular structural part of or the whole of the compound.

The terms “binds”, “able to bind to” or “capable of binding” as used herein means that under standard immunoassay conditions, for example as described in ‘Immunoassay: A practical guide’ by Brian Law, Taylor and Francis Ltd (ISBN 0-203-48349-9), the antibodies will bind to said molecules.

### Kits

The invention also describes a kit comprising an antibody of the invention and optionally a detecting agent of the invention which is preferably of structure II. Preferred kits are described in claims 16 and 17. The kit can also incorporate antibodies, derived from any of the immunogens of the invention, passively adsorbed on or chemically bonded to a solid state device.

### Substrates

A solid state device may also be referred to as a substrate. The antibodies engage with the substrate by, for example, passive adsorption or can be chemically bonded to the substrate attached by way of, for example, covalent bonds. Such covalent bonding generally requires the initial introduction of a chemically active compound covalently attached to the substrate surface prior to antibody addition. The antibody itself may also require the addition of a chemical activating group to achieve substrate bonding. These requirements are well known in the art. The substrate can be any medium capable of adsorbing or bonding to an antibody, for example a bead, a microtitre plate or a nanoparticle (all optionally chemically-activated), but is preferably of a planar conformation (optionally chemically-activated) such as a glass slide or a biochip. A biochip or microtitre plate is the preferred substrate. A biochip is a thin, wafer-like substrate with a planar surface which can be made of any suitable material such as glass or plastic but is preferably made of ceramic. The biochip is able to be chemically-activated prior to antibody bonding or is amenable to the passive adsorption of antibodies. The skilled person in biochip development for immunoassay application will recognize that a planar surface at high resolution e.g. if using a scanning electron microscope, is not perfectly ‘flat’ but will possess an uneven surface, the important aspect being that the ‘approximately’ planar surface is suitable for application. A microlayer coating of material can optionally be added to the planar surface of the substrate prior to antibody placement. Either the upper surface or both surfaces of the substrate can be coated. The biochip can be integrated into or placed into a device with walls. Such a walled device can aid in the retention of added sample or solution. The solid state device can also support other antibodies which have a binding specificity which is different from the binding specificity of the antibodies of the invention. Such a support with multiple different antibodies is often described as a multianalyte array (reference to an ‘array’ includes a microarray). If the method of detection is different fluorescent labels, each different fluorescent label emitting electromagnetic radiation at a unique wavelength, then the location of placement of the antibodies on the solid substrate is not critical. However, for antibodies forming part of a multianalyte array in which the detecting agent is, for example, a chemiluminescent molecule, the antibodies of differing specificity must not overlap and must be located in discrete areas on the solid state device. Such a system is also referred to as a spatially addressable multianalyte array.

### General Methods, Examples and Results

### Preparation of Haptens, Immunogens and Detecting Agents

In immunology, haptens are defined as substances which by themselves cannot elicit immune responses; they require chemical coupling to larger immunogenic molecules (antigenicity conferring carrier materials or ‘accm’), to be capable of inducing an immune response. Appropriate accms commonly contain poly(amino acid) segments and include polypeptides, proteins and protein fragments. Illustrative examples of antigencity conferring carrier materials are keyhole limpet haemocyanin (KLH), bovine thyroglobulin (BTG), bovine serum albumin (BSA), egg ovalbumin, bovine gamma globulin or cationised BSA. Alternatively, synthetic poly(amino acids) having a sufficient number of available amino groups, such as lysine, may be employed, as may other synthetic or natural polymeric materials bearing reactive functional groups. Also, carbohydrates, yeasts or polysaccharides may be conjugated to the hapten to produce an immunogen. The haptens can also be coupled to a detectable labelling agent such as an enzyme (for example, horseradish peroxidase), a substance having fluorescent properties or a radioactive label for the preparation of detecting agents for use in the immunoassays. The fluorescent substance may be, for example, a monovalent residue of fluorescein or a derivative thereof. Conjugation of haptens can be performed using standard methods of conjugation such as mixed anhydride, EDC or succinimidyl activation of the haptens. In order to confirm that adequate conjugation of hapten to carrier material has been achieved, prior to immunisation, each immunogen is evaluated using matrix-assisted UV laser desorption/ionisation time-of-flight mass spectroscopy (MALDI-TOF MS).

### General Procedure for MALDI-TOF Analysis of Immunogens

MALDI-TOF mass spectrometry was performed using a Voyager STR Biospectrometry Research Station laser-desorption mass spectrometer coupled with delayed extraction. An aliquot of each sample to be analysed was diluted in 0.1% aqueous trifluoroacetic acid (TFA) to create 1 mg/ml sample solutions. Aliquots (1 μl) were analysed using a matrix of sinapinic acid and bovine serum albumin (Fluka) was used as an external calibrant.

### Immunoassay Development

The process of developing an immunoassay is well known to the person skilled in the art. A detecting agent (e.g. appropriate hapten conjugated to HRP) is added to a sample containing the target analyte and the raised antibodies, and the detecting agent and analyte compete for binding to the antibodies. The said antibodies were fixed to a polystyrene solid support (e.g. dilution of antibodies in coating buffer and incubation at 37° C. for 2 hours to allow antibody binding to surface). The antibodies can be polyclonal or monoclonal using standard techniques, but the current invention makes use of polyclonal antibodies. The signal emitted in the immunoassay is proportionate to the amount of detecting agent bound to the antibodies which in turn is inversely proportionate to the analyte concentration. The signal can be detected or quantified by comparison with a calibrator with known levels of target analyte.

### EXAMPLE 1

### Synthesis of 2-(4-cyclohexylpiperazin-1-yl)-2-phenylacetonitrile (Compound 2 FIG. 2)

A mixture of H₂O (13 ml) and sodium bisulfite (5.14 g, 49.43 mmol) were mixed in a beaker, with stirring. Benzaldehyde 1 (5.24 g, 49.43 mmol) was added and the mixture was stirred for 30 mins, during which time a slurry of the benzaldehyde-bisulfite addition product formed. A 20% aq. solution of 1-cyclohexylpiperazine (8.5 g, 50.42 mmol) in H₂O (35 ml) was then added dropwise to the reaction mixture with stirring (during which most of the compound dissolved). After the addition was complete, sodium cyanide (2.42 g, 49.43 mmol) was added portion-wise over a period of 15 mins. The reaction mixture's beaker was covered and left stirring overnight. Ethyl acetate (200 ml) was added to the reaction mixture and the organic layer was separated and washed successively with water (150 ml) and brine (150 ml) (aqueous layers were combined and left stirring overnight with excess sodium hypochlorite to quench the remaining cyanide). The organic portion was dried over Na₂SO₄, filtered and concentrated to dryness in vacuo. The crude product obtained was purified by chromatography (Biotage, 80 g ZIP sphere cartridge, ethyl acetate (0→50%) in pet. ether to afford Compound 2. (10.25 g, 36.17mmol, 73% yield) as a white solid.

### EXAMPLE 2

### Synthesis of 1-cyclohexyl-4-(2-(3-methoxyphenyl)-1-phenylethyl)-piperazine (3-OMe MT-45) (Compound 3 FIG. 2)

In a flame dried 3-neck round bottom flask, with a condenser and a dropping funnel attached, under nitrogen, was added Magnesium (Mg) (5.27 g, 217 mmol), anhydrous diethyl ether (45 ml) and a crystal of Iodine. Under vigorous stirring, at room temperature, 20-30 drops of 3-methoxybenzyl bromide (total amount needed for the reaction: 14.54 g, 72.33 mmol) were added to the reaction mixture. After 1-2 minutes the iodine colour fades out and the reaction mixture starts to self-reflux. Then, through the dropping funnel, the rest of the 3-methoxybenzyl bromide, dissolved in anhydrous diethyl ether (45 ml), was added dropwise to the reaction mixture at such a rate that the self-reflux is maintained. After the addition completed, the flask was heated at reflux for 1 h. Then, the reaction mixture was cooled down to RT and 2-(4-cyclohexylpiperazin-1-yl)-2-phenylacetonitrile 2 (10.25 g, 36.17mmol) dissolved in anhydrous diethyl ether (45 ml) was added dropwise. When the addition was completed, the reaction mixture was heated to reflux overnight. The reaction mixture was then cooled to RT and a saturated aq. solution of ammonium chloride (˜200 ml) was cautiously added and stirred for 1 h. The mixture was extracted with ethyl acetate (2×250 ml). The combined organic extracts were dried over Na2SO4, filtered and concentrated in vacuo to dryness. The crude product obtained was purified by chromatography (Biotage, 80 g ZIP sphere cartridge, ethyl acetate (0→80%) in pet. ether) (Caution: The product has a low UV activity) to afford Compound 3 (13.5 g, 35.67 mmol, 98% yield).

### EXAMPLE 3

### Synthesis of 3-(2-(4-cyclohexylpiperazin-1-yl)-2-phenylethyl) phenol (3-Hydroxy MT-45) (Compound 4 FIG. 2)

1-cyclohexyl-4-(2-(3-methoxyphenyl)-1-phenylethyl)piperazine (13.5 g, 35.67 mmol) (compound 3) was dissolved in acetic acid (100 ml) and HBr 48% (400 ml), in a round bottom flask, and the mixture was refluxed overnight. The next morning the solution had turned black. The reaction mixture was poured into ice (300 g) and, carefully, ammonium hydroxide, 28-30% solution, was added until alkaline (pH=10). Then, DCM (1 L was added and the organic layer was separated, washed by brine (250 ml), dried over Na2SO4, filtered and concentrated in vacuo to dryness, affording Compound 4 as a brownish solid (8.0 g, 21.94 mmol, yield=62%), which was used crude in Example-4.

### EXAMPLE 4

### Synthesis of tert-butyl 2-(3-(2-(4-cyclohexylpiperazin-1-yl)-2-phenylethyl)phenoxy) acetate (Compound 5 FIG. 2)

3-(2-(4-cyclohexylpiperazin-1-yl)-2-phenylethyl) phenol (compound 4) (3.0 g, 8.24 mmol) was dissolved in anhydrous DMF (40 ml) under nitrogen and sodium hydride (60% suspension in mineral oils) (495 mg, 12.36 mmol) was added at 0oC. After 1h of stirring in the ice bath (gas evolution had ceased), t-butyl bromoacetate (1.46 ml, 9.89 mmol) was added and the reaction mixture was kept at 0 oC for 30 mins and allowed to warm to RT. After stirring for 1 h at RT, a few drops of water were carefully added to quench the excess of NaH. The volatiles were removed in vacuo and ethyl acetate (200 ml) and saturated aqueous solution of NH4Cl (150 ml) were added to the crude residue. The organic layer was separated, dried over Na2SO4, filtered and concentrated in vacuo. The crude product obtained was purified by chromatography (Biotage, 45 g ZIP sphere cartridge, EtOAc (0→70%) in pet. ether) to afford the ester (Compound 5) (2.8 g, 5.86 mmol, 71% yield).

### EXAMPLE 5

### Synthesis of 2-(3-(2-(4-cyclohexylpiperazin-1-yl)-2-phenylethyl) phenoxy)acetic acid (MT-45-3-CME, (Compound 6 Hapten A FIG. 2)

Tert-butyl 2-(3-(2-(4-cyclohexylpiperazin-1-yl)-2-phenylethyl) phenoxy) acetate (2.8 g, 5.86 mmol) was mixed with DCM (40 ml) and TFA (20 ml). The mixture was stirred at RT overnight. The volatiles were removed in vacuo to dryness and the crude product obtained was purified by chromatography (Biotage, 45 g ZIP sphere cartridge, MeOH (0→30%) in ethyl acetate) to afford Compound 6 (1.50 g, 3.55 mmol, 61% yield) as a white solid.

### EXAMPLE 6

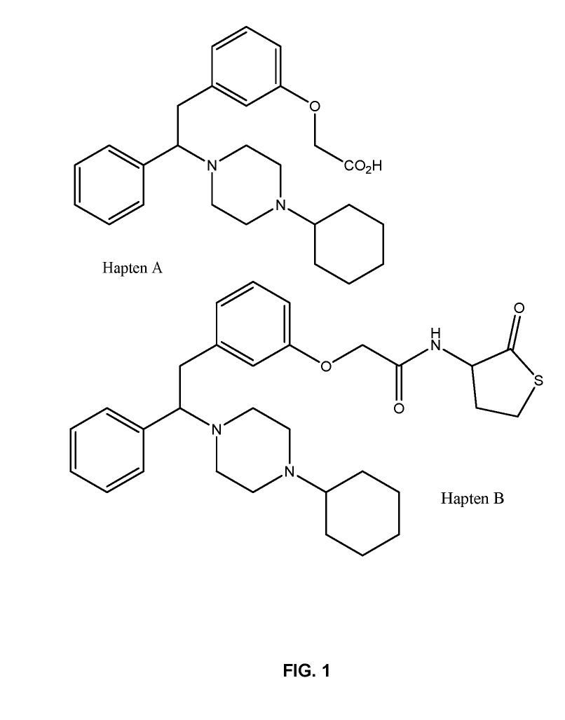

### Synthesis of 3-[2-(4-cyclohexylpiperazin-1-yl)-2-phenylethyl)] phenoxy-N-(2-oxotetrahydrothiophen-3-yl)acetamide (Hapten B FIG. 1)

2-(3-(2-(4-Cyclohexylpiperazin-1-yl)-2-phenylethyl) phenoxy) acetic acid (compound 6 Hapten A FIG. 2) (500 mg, 1.18 mmol) was dissolved in pyridine (16 ml). DL-homocysteine thiolactone hydrochloride (273 mg, 1.77 mmol) was added, followed by EDCI.HCl (341 mg, 1.77 mmol). The reaction mixture was left to stir overnight at RT. The volatiles were removed in vacuo and the crude product obtained was purified by chromatography (Biotage via a 30 g ZIP sphere cartridge, MeOH (0→20%) in ethyl acetate) to afford the titled product (420 mg, 0.81 mmol, 68% yield) as an orange solid.

### EXAMPLE 7

### Conjugation of 2-(3-(2-(4-cyclohexylpiperazin-1-yl)-2-phenylethyl) phenoxy) acetic acid (MT-45-3-CME (Compound 6Hapten A FIG. 2). to BSA (Immunogen 1)

To a solution of MT-45-3-CME (Hapten A) (32.5 mg) in DMF (1.0 ml) was added N-(3-dimethylaminopropyl)-N′-ethylcarbodiimide hydrochloride (EDC.HCl) (73.8 mg) and N-hydroxysuccinimide (44.2 mg) and the mixture was incubated on the roller at room temperature overnight. This solution was added dropwise to a solution of BSA (100 mg, 1.5 micomol) in phosphate buffer saline (50 mM) (pH 8.0) (10 ml). The resulting solution was incubated on the roller at room temperature overnight. Excess hapten was removed by dialysis against phosphate buffer saline, pH 7.2 (3 changes) for 24 hours at 4° C., and freeze-dried. MALDI results showed 26.5 molecules of MT-45-3-CME (Hapten A) had been conjugated to one molecule of BSA.

### EXAMPLE 8

### Conjugation of 2-(3-(2-(4-cyclohexylpiperazin-1-yl)-2-phenylethyl)phenoxy)acetic acid (MT-45-3-CME (Compound 6Hapten A FIG. 2). to KLH (Immunogen 2)

To a solution of MT-45-3-CME (Hapten A) (32.5 mg) in DMF (1.0 ml) was added N-(3-dimethylaminopropyl)-N′-ethylcarbodiimide hydrochloride EDC. HCl (73.7 mg) and N-hydroxysuccinimide (44.3 mg) and the mixture was incubated on the roller at room temperature overnight. This solution was added dropwise to a solution of KLH (100 mg) in phosphate buffer saline (50 mM) (pH 8.0) (10 ml). The resulting solution was incubated on the roller at room temperature overnight. Excess hapten was removed by dialysis against phosphate buffer saline, pH 7.2 (3 changes) for 24 hours at 4° C., and freeze-dried.

### EXAMPLE 9

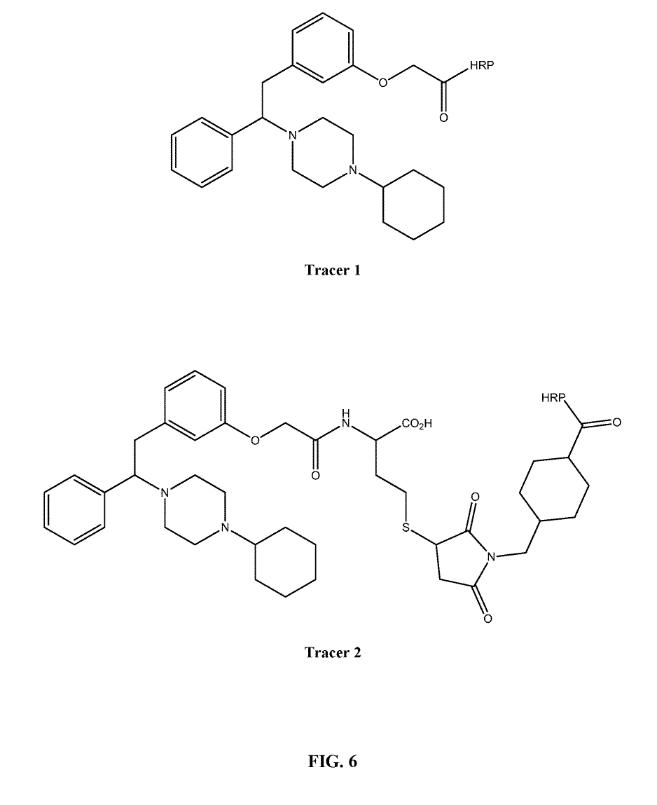

### Conjugation of 2-(3-(2-(4-cyclohexylpiperazin-1-yl)-2-phenylethyl) phenoxy) acetic acid (MT-45-3-CME, (Compound 6 Hapten A FIG. 2) to HRP (Tracer 1 FIG. 6)

EDC hydrochloride (1.5 mg) was dissolved in water (0.5 ml) and immediately added to a solution of MT-45-3-CME (Hapten A) (3.0 mg) in DMF (0.2 ml). After mixing, this solution was added dropwise to a solution of HRP (20 mg) in water (1 ml.).

N-hydroxysuccinimide (1 mg) was added and the reaction mixture was incubated in the dark at room temperature overnight. Excess hapten was removed with double PD-10 columns (Pharmacia) in series, pre-equilibrated with PBS at pH 7.2.

The MT-45-3-CME-HRP conjugate (tracer 1) was then dialysed overnight against 10 L of PBS at pH 7.2 at 4° C.

### EXAMPLE 10

### Conjugation of 2-(3-(2-(4-cyclohexylpiperazin-1-yl)-2-phenylethyl) phenoxy)-N-(2-oxotetrahydrothiophen-3-yl) acetamide (MT-45-3-CM E-HCTL) (Hapten B FIG. 1) to HRP-maleimide.(Tracer 2 FIG. 6)

Preparation of HRP-maleimide: 4-(N-maleimidomethylcyclohexyl)-1-carboxylic acid NHS ester (0.84 mg) in DMF (0.045 ml) was added drop-wise to the HRP (20 mg) dissolved in 50 mM HEPES solution, pH8.5 (0.8 ml) while stirring. The resulting solution was stirred at 15-25° C. for 40 minutes. Excess maleimide linker was removed by dialysis against phosphate buffer saline pH7.2. Pre-activation of the hapten: MT-45 3-CME HCTL (Hapten-B) (2 mg) was dissolved in DMF (0.2 ml). To this solution was added potassium hydroxide (1M) (0.2 ml) and the solution was incubated for 10 minutes. 0.5 ml of 0.2M Phosphate buffer, Ph7.0 was added to quench reaction and 0.1M HCl solution added to neutralise the solution.

Conjugation: The HRP-maleimide was added to the activated hapten, rolled for two hours at room temperature and then cooled at 2 to 4° C. and rolled overnight. Excess hapten was removed with PD-10 column (Pharmacia), pre-equilibrated with PBS pH 7.2 and dialysed at 2-8° C. against PBS pH 7.2 to afford MT-45-CME-HCTL-maleimide-HRP (Tracer 2).

### EXAMPLE 11

### Preparation of Antisera

Pre-immunization blood samples were collected from young adult, female, Texel sheep. In order to generate polyclonal antisera, 2 mgs of the immunogen (Immunogens 1 or 2) was prepared in PBS, mixed at a ratio of 50% immunogen in PBS to 50% Freund's Complete adjuvant (Sigma, Product Number F5881) and emulsified by repeatedly passing the mixture through a tip on the end of a 1 ml syringe, until the required semi-solid consistency was acquired. 1 ml of the emulsified mixture was injected intramuscularly into each host animal (sheep) as the primary immunisation dose. Further injections (boosts) were prepared (1 mg of immunogen in PBS and mixed at a ratio of 50% Immunogen in PBS/50% Freund's Incomplete adjuvant, Sigma, Product Number—F5506). Boost injections were delivered intramuscularly at monthly intervals, 1 ml per animal. Serum was sampled monthly by collection of whole blood from the jugular vein for evaluation of the antibody titre. Further purification of the serum with caprylic acid/ammonium sulphate precipitation was effected.

### EXAMPLE 12

### Competitive Assay for Cross-Reactivity Measurement

A 96 well ELISA plate was coated with 125 μl/well of antibody derived from Immunogen 2 (Example 8) at an appropriate concentration prepared in Tris buffer and was incubated at 4° C. for overnight. Contents were tipped out and plate washed (×4) using TBST before 50 μl of appropriate cross-reactant was added at various concentrations from 0-10ng/ml. 75 μl of tracer (Example 10—Tracer 1) was added to each well at an appropriate dilution, followed by incubation at 25° C. for 1 hour. Contents were tipped out and plate washed (×6) using TBST and 125 μl/well TMB solution was added. After 20 mins in dark, 125 μl/well 0.2M sulphuric acid was added. The plate was read at 450 nm using KC junior software. The cross-reactivity (CR) was calculated using the equation below. All calculations were based upon binding and displacement at the 50% of maximum OD (optical density) binding point. The maximum OD is the signal generated using tracer alone and 50% displacement (inhibition) corresponds to the IC₅₀.

Tables 1a-1c

Ave OD=average optical density

CV=coefficient of variation of OD

B=absorbance at 450 nm at xng/ml standard concentration

B₀=absorbance at 450 nm at 0 ng/ml standard concentration

IC₅₀=standard concentration which produces 50% inhibition of maximum signal

B/B0=(B/B0)×100%

CR=cross-reactivity (OD without cross-reactant−OD with cross-reactant)×100

Results

TABLE 1a

| Conc. | MT-45 | | | 3-Hydroxy MT-45 | | |
|---|---|---|---|---|---|---|
| ng/ml | Ave OD | % CV | B/Bo | Ave OD | % CV | B/Bo |
| 0 | 2.098 | 5.8 | 100 | 2.078 | 1.1 | 100 |
| 0.015625 | 1.903 | 3.6 | 91 | 1.905 | 3.3 | 92 |
| 0.03125 | 1.602 | 4.2 | 76 | 1.818 | 1.1 | 87 |
| 0.0625 | 1.506 | 3.2 | 72 | 1.511 | 3.2 | 73 |
| 0.125 | 1.069 | 8 | 51 | 1.203 | 6 | 58 |
| 0.25 | 0.687 | 5.8 | 33 | 0.765 | 7.2 | 37 |
| 0.5 | 0.339 | 4.3 | 16 | 0.389 | 9.3 | 19 |
| 1 | 0.165 | 0.3 | 8 | 0.192 | 9.2 | 9 |

| | | |
|---|---|---|
| IC50 | 0.131 ng/ml | 0.158 ng/ml |
| % CR | 100 | 82.9 |

TABLE 1b

| Conc. | MT-45 | | | 3-Hydroxy MT-45 | | |
|---|---|---|---|---|---|---|
| ng/ml | Ave OD | % CV | B/Bo | Ave OD | % CV | B/Bo |
| 0 | 1.945 | 1.7 | 100 | 1.925 | 1.9 | 100 |
| 0.03125 | 1.594 | 1.6 | 82 | 1.713 | 2.7 | 89 |
| 0.0625 | 1.543 | 2.7 | 79 | 1.573 | 2.2 | 82 |
| 0.125 | 1.286 | 2.9 | 66 | 1.353 | 0.8 | 70 |
| 0.25 | 1.006 | 3.5 | 52 | 1.101 | 1.6 | 57 |
| 0.5 | 0.728 | 4.3 | 37 | 0.811 | 2.3 | 42 |
| 1 | 0.462 | 1.9 | 24 | 0.528 | 0.7 | 27 |
| 2 | 0.276 | 2.8 | 14 | 0.321 | 2.4 | 17 |

| | | |
|---|---|---|
| IC50 | 0.279 ng/ml | 0.344 |
| % CR | 100 | 81.1 |

TABLE 1c

| Conc. | MT-45 | | | 3-Hydroxy MT-45 | | |
|---|---|---|---|---|---|---|
| ng/ml | Ave OD | % CV | B/Bo | Ave OD | % CV | B/Bo |
| 0 | 1.870 | 3.2 | 100 | 1.862 | 6.8 | 100 |
| 0.03125 | 1.549 | 3 | 83 | 1.725 | 1.5 | 93 |
| 0.0625 | 1.523 | 0.5 | 81 | 1.548 | 1.6 | 83 |
| 0.125 | 1.205 | 0.5 | 64 | 1.266 | 3.5 | 68 |
| 0.25 | 0.851 | 1.1 | 46 | 0.927 | 4.4 | 50 |
| 0.5 | 0.527 | 1.3 | 28 | 0.612 | 5 | 33 |
| 1 | 0.294 | 1.4 | 16 | 0.350 | 4 | 19 |
| 2 | 0.155 | 3.9 | 8 | 0.189 | 5 | 10 |

| | | |
|---|---|---|
| IC50 | 0.221 ng/ml | 0 250 ng/ml |
| % CR | 100 | 88.4 |

Table 1a—antisera at 0.625 μg/ml from sheep 1

Table 1a—3 replicate runs using 8 standard concentrations (provides CC values for each standard concentration) enabling derivation of 3×IC50 values and thus 3×CR values

3-Hydroxy MT-45

Curve 1: CR=75.8%

Curve 2: CR=80.5%

Curve 3: CR=92.4%

Average=82.9%

Table 1b—antisera at 10.00 μg/ml from sheep 2

Table 1b-3 replicate runs using 8 standard concentrations (provides CV values for each standard concentration) enabling derivation of 3×IC50 values and thus 3×CR values

3-Hydroxy MT-45

Curve 1: CR=73.4%

Curve 2: CR=82.6%

Curve 3: CR=87.3%

Average=81.1%

Table 1c—antisera at 5.00 μg/ml from sheep 3

### STATEMENTS OF THE INVENTION

1. An immuogen of structure

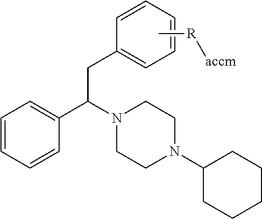

wherein R is a spacing group attached to the ortho, meta or para position of the phenyl ring and to an antigenicity conferring carrier material (accm).

2. The immunogen of statement 1 in which R is attached to the meta or para position of the phenyl ring.

3. The immunogen of statement 2 in which R is -(Q)m-Y—X—, in which Q or Y (if Q is not present) is attached to the meta or para position of the phenyl ring and m=0 or 1; Y can comprise a substituted or unsubstituted, straight or branched chain, saturated or unsaturated alkanediyl moiety, a substituted or unsubstituted, saturated or unsaturated cycloalkylene moiety or a substituted or unsubstituted arylene moiety; X, before attachment to the antigenicity conferring carrier material, is chosen from carboxy, dithiopyridyl, maleimidyl, amino, hydroxyl, thiol, thioester, or aldehyde; Q is —O—, —S—, —NH—, —C(O)—, —C(O)—O—., —S(O)—, —S(O)₂—, —C(S)—O— or —C(O)—S—.

4. The immunogen of statement 3 in which m=1 and Q is —O—, —NH— or —S—, and Y is a C₁₋₆ substituted or unsubstituted, straight chain, saturated alkanediyl moiety.

5. The immunogen of statement 4 in which Q is attached to the meta position of the phenyl ring.

6. The immunogen of any of the preceding statements in which the antigenicity conferring carrier material is keyhole limpet haemocyanin, bovine thyroglobulin, bovine serum albumin, egg ovalbumin, bovine gamma globulin or cationised BSA.

7. An antibody derived from an immunogen of any of statements 2 to 6 which binds to MT-45.

8. The antibody of statement 7 derived from an immunogen in which Q is —O—, Y is —CH₂—, X is carboxy and the accm is keyhole limpet haemocyanin which has an IC₅₀ of <1.00 ng/ml to MT-45 as calculated from a standard curve using MT-45-CME-HCTL-maleimide-HRP (Tracer 2) as detecting agent.

9. An immunoassay method of detecting or determining MT-45 in an in vitro sample or in a solution comprising contacting the sample or solution with a detecting agent and an antibody of statements 7 or 8, detecting the bound detecting agent and deducing the presence or amount of MT-45.

10. The immunoassay method of statement 9 wherein the detecting agent is

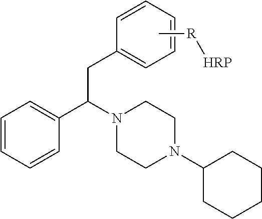

wherein R of the detecting agent is a crosslinking group attached to the meta or para position of the phenyl ring and HRP is horseradish peroxidase.

11. The immunoassay method of statement 10 wherein R of the detecting agent is attached to the meta position of the phenyl ring.

12. A detecting agent of structure

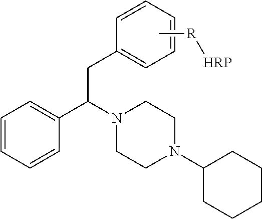

wherein R of the detecting agent is a crosslinking group attached to the meta or para position of the phenyl ring and HRP is horseradish peroxidase.

13. The detecting agent of statement 12 which has the structure

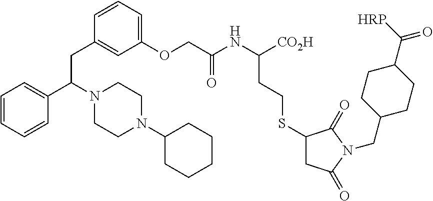

14. A kit comprising an antibody of either of statements 7 or 8 and optionally a detecting agent as described in claim 13.

What is claimed is:

1. An immuogen of structure 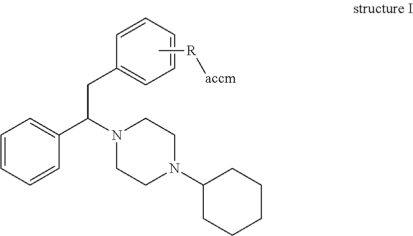
wherein:
R is a spacing group attached to the ortho, meta or para position of the phenyl ring and to an antigenicity conferring carrier material (accm).

2. The immunogen of claim 1, wherein R is attached to the meta or para position of the phenyl ring.

3. The immunogen of claim 2, wherein:
R is -(Q)m-Y—X—, wherein:
Q or Y (if Q is not present) is attached to the meta or para position of the phenyl ring;
m=0 or 1;
Y is a substituted or unsubstituted, straight or branched chain alkanediyl moiety, a substituted or unsubstituted, saturated or unsaturated cycloalkylene moiety, or a substituted or unsubstituted arylene moiety;
X, before attachment to the antigenicity conferring carrier material, is carboxy, dithiopyridyl, maleimidyl, amino, hydroxyl, thiol, thioester, or aldehyde; and
Q is —O—, —S—, —NH—, —C(O)—, —C(O)—O—., —S(O)—, —S(O)₂—, —C(S)—O— or —C(O)—S—.

4. The immunogen of claim 3, wherein:
m=1;
Q is —O—, —NH— or —S—; and
Y is a C₁₋₆ substituted or unsubstituted, straight chain, saturated alkanediyl moiety.

5. The immunogen of claim 4, wherein Q is attached to the meta position of the phenyl ring.

6. The immunogen of claim 1, wherein the antigenicity conferring carrier material is keyhole limpet haemocyanin, bovine thyroglobulin, bovine serum albumin, egg ovalbumin, bovine gamma globulin or cationised BSA.

7. An antibody raisable from an immunogen of claim 2.

8. The antibody of claim 7, wherein the antibody binds to MT-45.

9. The antibody of claim 8, wherein the antibody is raisable against an immunogen of structure III: 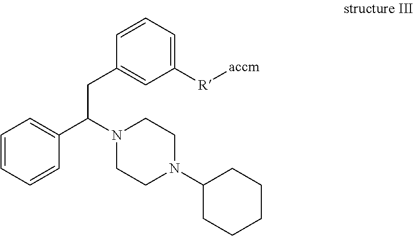
wherein:
R′ is -(Q′)ₘ-Y′—X′
m is 1
Q′ is —O—;
Y′ is —CH₂—;
X′ is carboxy; and
the accm is keyhole limpet haemocyanin
wherein the antibody has an IC₅₀ of <1.00 ng/ml to MT-45 as calculated from a standard curve using MT-45-CME-HCTL-maleimide-HRP (Tracer 2) as detecting agent.

10. An immunoassay method of detecting or determining MT-45 in an in vitro sample or in a solution comprising:
contacting the sample or solution with a detecting agent and an antibody of claim 7;
detecting the amount of detecting agent bound to the antibody; and
deducing the presence or amount of MT-45.

11. The immunoassay method of claim 10, wherein the detecting agent is: 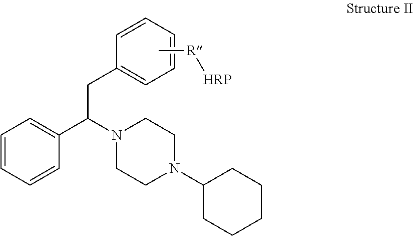
wherein:
R″ is a crosslinking group attached to the meta or para position of the phenyl ring and HRP is horseradish peroxidase.

12. The immunoassay method of claim 11, wherein R″ is attached to the meta position of the phenyl ring.

13. The immunoassay method of claim 12, wherein the antibody has a cross-reactivity of between about 60% to about 95% for 3-hydroxy MT-45 compared with a cross-reactivity of about 100% to MT-45.

14. The immunoassay method of claim 13, wherein the antibody has a cross-reactivity of between about 70% to about 95% for 3-hydroxy MT-45 compared with a cross-reactivity of about 100% to MT-45.

15. The immunoassay method of claim 14, wherein the antibody has a cross-reactivity of between about 75% to about 90% for 3-hydroxy MT-45 compared with a cross-reactivity of about 100% to MT-45.

16. A detecting agent of structure 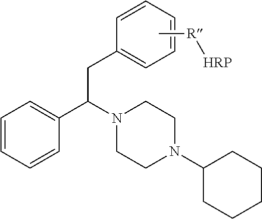
wherein:
R″ is a crosslinking group attached to the meta or para position of the phenyl ring and HRP is horseradish peroxidase.

17. The detecting agent of claim 16, which has the structure

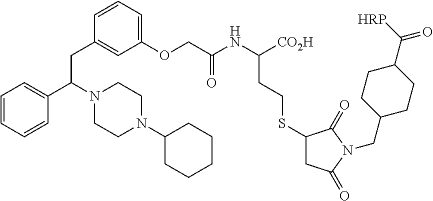

18. A kit comprising an antibody of claim 7.

19. The kit of claim 18, further comprising the detecting agent of claim 17.
